(19) United States
(12) Patent Application Publication
(10) Pub. No.: US 2012/0197220 A1
(43) Pub. Date: Aug. 2, 2012

(54) GAS DISPENSING DEVICE FOR MEDICAL USE

(76) Inventor: YU-KANG HUANG, TAICHUNG CITY (TW)

(21) Appl. No.: 13/014,844

(22) Filed: Jan. 27, 2011

(51) Int. Cl. A61F 13/00

(52) U.S. Cl. 604/305

(57) ABSTRACT

A gas dispensing device for medical use includes a hollow bag and an inlet hose, or further includes a support member. The bag has a room defined therein and includes a dispensing wall. Multiple holes are defined through the dispensing wall and communicate with the room. The inlet hose is inserted into the room of the hollow bag and the hollow bag is supported by the support member. The device provides gas to the wounded area of patients.

### FIELD OF THE INVENTION

The present invention relates to a gas dispensing device for medical use, and more particularly, to a gas dispensing device for medical use having multiple holes for continuously providing gas.

### BACKGROUND OF THE INVENTION

One of conventional methods for taking care of large area burns or scalds, or necrotizing cellulites is the oxygen therapy. The wounded areas usually are combined with infection of aerobic and anaerobic necroses which result in hypoxia to the cells of the wounded area. The oxygen therapy provides sufficient oxygen to the capillary of the wounded area to increase blood circulation and provide nutrition to the cells to deal with the edema and hypoxia, and retrieve normal aerobic metabolism.

The oxygen therapy usually provides the patient under 2 to 2.5 atmosphere of pure oxygen for 90 to 120 minutes and the therapy keeps at least 30 days. One of the devices for the oxygen therapy is disclosed in Taiwan Utility model Patent No. M360028 and includes a tank which is suitable for the user to operate and includes an oxygen volume valve, an air switch, an oxygen switch, an auto/manual pressure release valve, and a manual air release valve. The device allows the user to be treated in a highly concentrated oxygen surrounding to have benefits of health care and beauty care.

However, the device of oxygen therapy is expensive and usually designed to taking care of large wounded area. Therefore, the present invention provides a medical use gas dispensing device for small wounded area.

### SUMMARY OF THE INVENTION

The present invention relates to a gas dispensing device for medical use and comprises a hollow bag and an inlet hose. The hollow bag has a room defined therein. The hollow bag comprises a dispensing wall and a sealing wall. Multiple holes are defined through the dispensing wall. The inlet hose is connected to the hollow bag and communicates with the room.

The hollow bag comprises a first connection portion on a periphery thereof and a support member has a second connection portion which is connected with the first connection portion by two respective fastening members respectively provided on the first and second connection portions. The fastening members are loop-and-hook strips, double sided tapes or clips. The support member is a C-shaped member and includes an opening. The second connection portion is located at an inner periphery of the support member. The hollow bag is located within the inner periphery of the support member and the inlet hose extends through the opening. The height of cross section of the support member is higher than the height of cross section of the hollow bag. The hollow bag is located within the height of the support member.

Alternatively, the gas dispensing device for medical use comprises a hollow bag and an inlet hose. The hollow bag has a room defined therein. The hollow bag comprises a dispensing wall and a sealing wall. Multiple holes are defined through the dispensing wall. The inlet hose is connected to the hollow bag and communicates with the room. A support member is integrally formed with the hollow bag. The hollow bag comprises a first connection portion on a periphery thereof and the support member has a second connection portion which is connected with the first connection portion.

The primary object of the present invention is to provide a gas dispensing device for medical use which is used for small wounded area and conveniently operated.

The gas dispensing device for medical use of the present invention is conveniently and easily operated by connecting an oxygen tank or an oxygen supply apparatus to the inlet hose, keeping providing the wounded area with oxygen.

The hollow bag of the gas dispensing device for medical use is supported on the support member and the underside of the support member is rested around the wounded area so that the operation is easy and the patient is comfortable.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view to show the gas dispensing device for medical use of the present invention;

FIG. 2 is a perspective view to show the gas dispensing device for medical use of the present invention;

FIG. 3 is a cross-sectional view of the gas dispensing device for medical use of the present invention;

FIG. 4 shows that the inlet hose of the gas dispensing device for medical use is connected to an oxygen tank;

FIG. 5 is a cross sectional view to show the operation of the gas dispensing device for medical use of the present invention, and

FIG. 6 shows that the hollow bag and the support member are integrally formed to each other.

### DETAILED DESCRIPTION OF THE PREFERRED EMBODIMENT

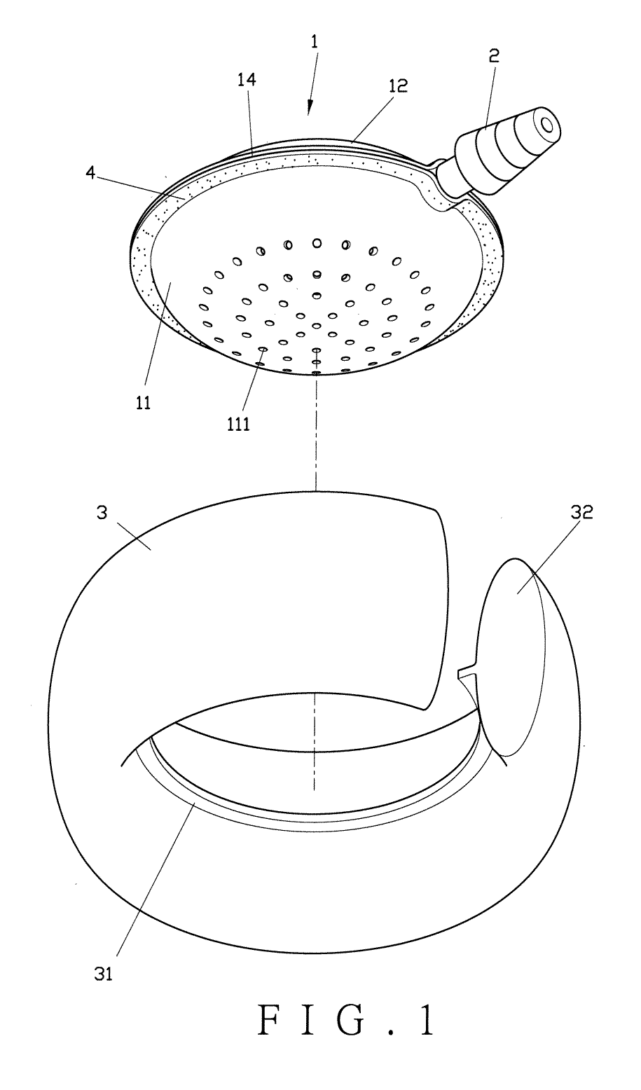

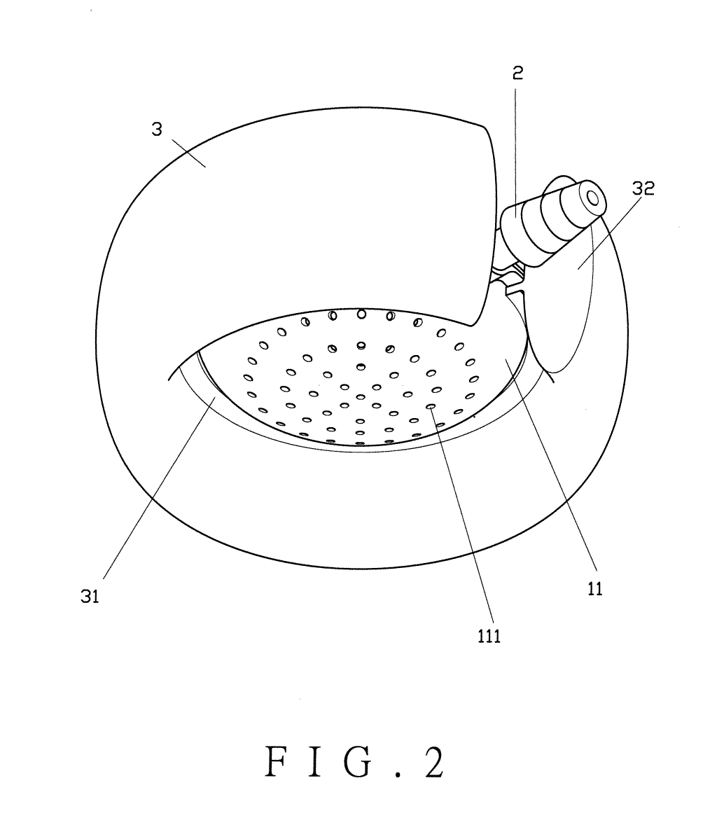

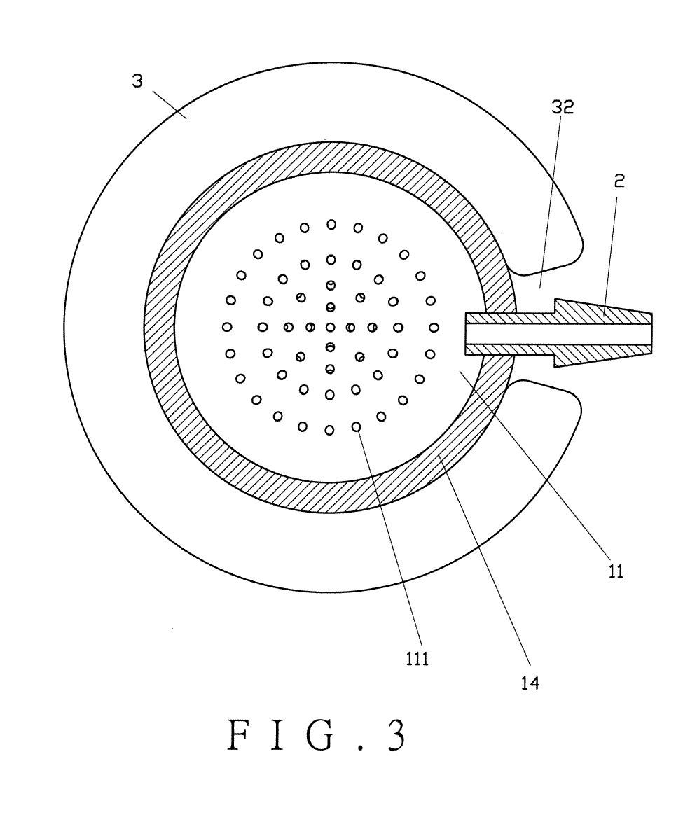

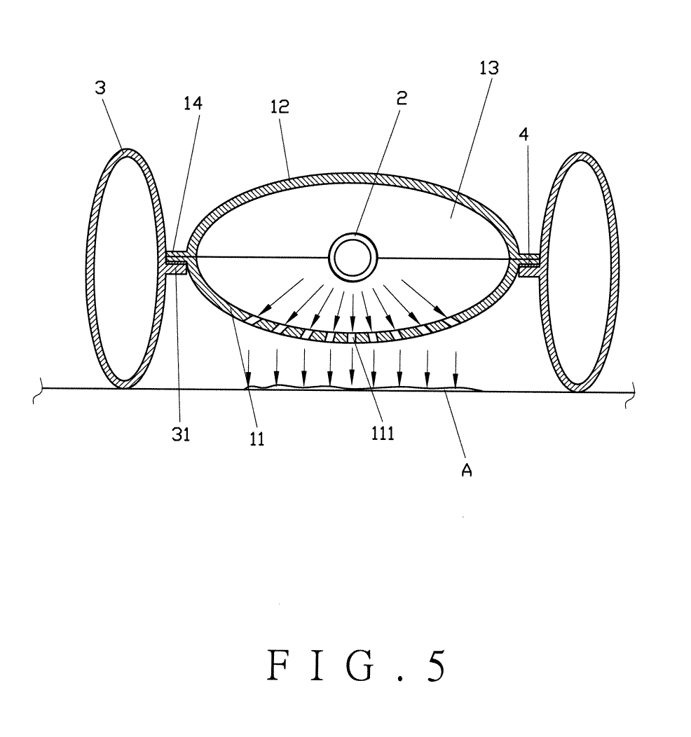

Referring to FIGS. 1 to 3, the gas dispensing device for medical use of the present invention comprises a hollow bag 1 and an inlet hose 2. The hollow bag 1 comprises a dispensing wall 11 and a sealing wall 12 which is connected to the dispensing wall 11 by way of high frequency fusion. The hollow bag 1 has a room 13 defined therein as shown in FIG. 5. Multiple holes 111 are defined through the dispensing wall 11 and communicate with the room 13. The hollow bag 1 comprises a first connection portion 14 on a periphery thereof and the first connection portion 14 is a plate-like part that is reserved when making the hollow bag 11 by way of high frequency fusion. The inlet hose 2 is connected to the hollow bag 1 and communicates with the room 13.

In order to provide easy operation to the users and comfort to the patients, the gas dispensing device for medical use further comprises a support member 3 which is an annular bag filled with foam or an inflated bag. The support member 3 has a second connection portion 31 which is located at an inner periphery thereof and connected with the first connection portion 14. Two respective fastening members 4 are respectively provided on the first and second connection portions 14, 31 so as to connect the first and second connection portions 14, 31. The fastening members 4 are loop-and-hook strips, double sided tapes or clips. The height of cross section of the support member 3 is higher than the height of cross section of the hollow bag 1, so that the hollow bag 1 is located within the height of the support member 3. The support member 3 is a C-shaped member and includes an opening 32. The inlet hose 2 extends through the opening 32.

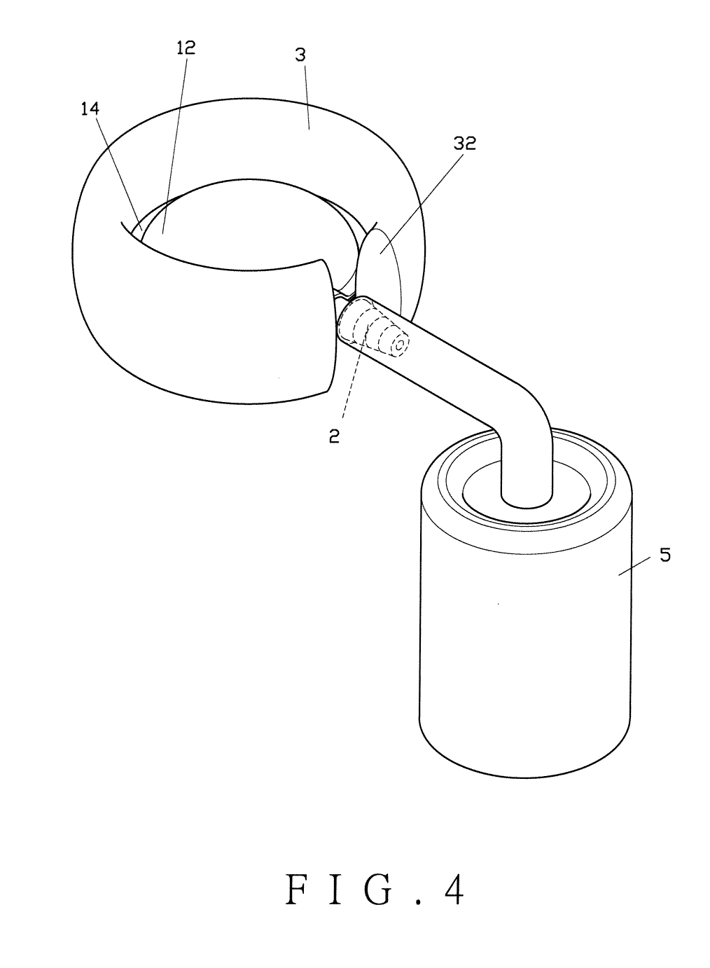

As shown in FIGS. 4 and 5, when in use, the support member 3 is rested around the wounded area “A” and the holes 111 of the hollow bag 1 face the wounded area “A”. The inlet hose 2 is connected to an oxygen supply apparatus or a tank 5 which includes high pressurized oxygen received therein. The oxygen enters the room 13 and blows toward the wounded area “A” via the holes 111 to treat the wounded area “A”.

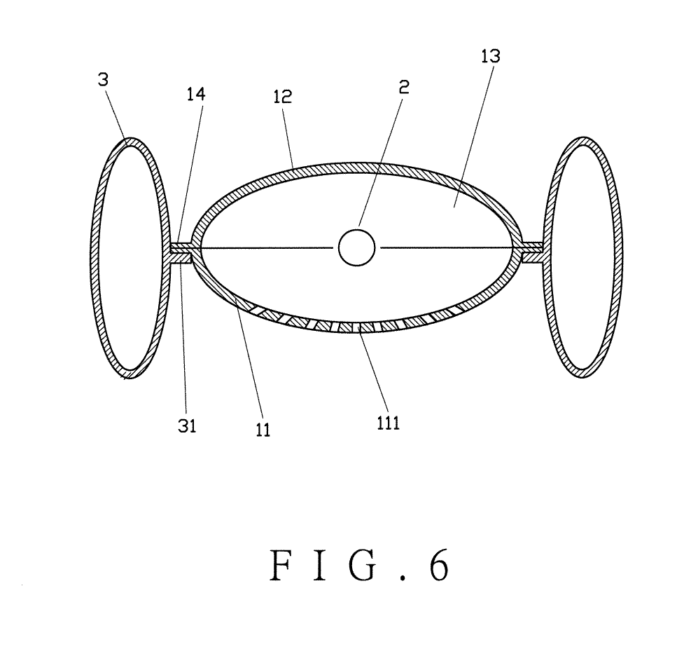

The second embodiment of the present invention is shown in FIG. 6 and the difference from the first embodiment is that the second connection portion 31 of the support member 3 is integrally formed with the first connection portion 14 of the hollow bag 1. The hollow bag 1 is made by the same method as the first embodiment and the first and second connection portions 14, 31 are connected to each other by way of high frequency fusion to integrally connect the hollow bag 1 and the support member 3. The way of operation is the same as the first embodiment.

The manufacturing of the hollow bag 1 and the annular support member 3, regardless of two separated parts or one-piece part, is easy and can be made at low cost.

While we have shown and described the embodiment in accordance with the present invention, it should be clear to those skilled in the art that further embodiments may be made without departing from the scope of the present invention.

What is claimed is:

1. A gas dispensing device for medical use, comprising:
a hollow bag having a room defined therein, multiple holes defined through the hollow bag and communicating with the room, and
an inlet hose connected to the hollow bag and communicating with the room.

2. The device as claimed in claim 1, wherein the hollow bag comprises a dispensing wall and a sealing wall, and the holes are defined through the dispensing wall.

3. The device as claimed in claim 2, further comprising a support member, the hollow bag having a first connection portion on a periphery thereof, the support member having a second connection portion which is connected with the first connection portion.

4. The device as claimed in claim 3, wherein two respective fastening members are respectively provided on the first and second connection portions so as to connect the first and second connection portions.

5. The device as claimed in claim 4, wherein the fastening members are loop-and-hook strips, two-sided tapes or clips.

6. The device as claimed in claim 4, wherein the support member is a C-shaped member and includes an opening, the second connection portion is located at an inner periphery of the support member, the hollow bag is located within the inner periphery of the support member and the inlet hose extends through the opening, a height of cross section of the support member is higher than a height of cross section of the hollow bag, and the hollow bag is located within the height of the support member.

7. A gas dispensing device for medical use, comprising:
a hollow bag having a room defined therein, multiple holes defined through the hollow bag and communicating with the room;
an inlet hose connected to the hollow bag and communicating with the room, and
a support member integrally formed with the hollow bag.

8. The device as claimed in claim 7, wherein the hollow bag comprises a dispensing wall and a sealing wall, the holes are defined through the dispensing wall, the hollow bag comprises a first connection portion on a periphery thereof, and the support member has a second connection portion which is connected with the first connection portion.

9. The device as claimed in claim 8, wherein the support member is a C-shaped member and includes an opening, the second connection portion is located at an inner periphery of the support member, the hollow bag is located within the inner periphery of the support member, and the inlet hose extends through the opening.

10. The device as claimed in claim 9, wherein a height of cross section of the support member is higher than a height of cross section of the hollow bag, and the hollow bag is located within the height of the support member.
